(19) United States
(12) Patent Application Publication
(10) Pub. No.: US 2004/0198723 A1
(43) Pub. Date: Oct. 7, 2004

(54) Method of treatment

(76) Inventor: Karen Gibson, Sheffield (GB)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/053,962

(22) Filed: Jan. 22, 2002

(51) Int. Cl. A61K 31/5513; A61K 31/485

(52) U.S. Cl. 514/221; 514/282

(57) ABSTRACT

There is described a method of treatment of a patient undergoing opioid analgesic therapy which comprises minimising the side effects of the opioid by the administration of a therapeutically effective amount of devazepide.

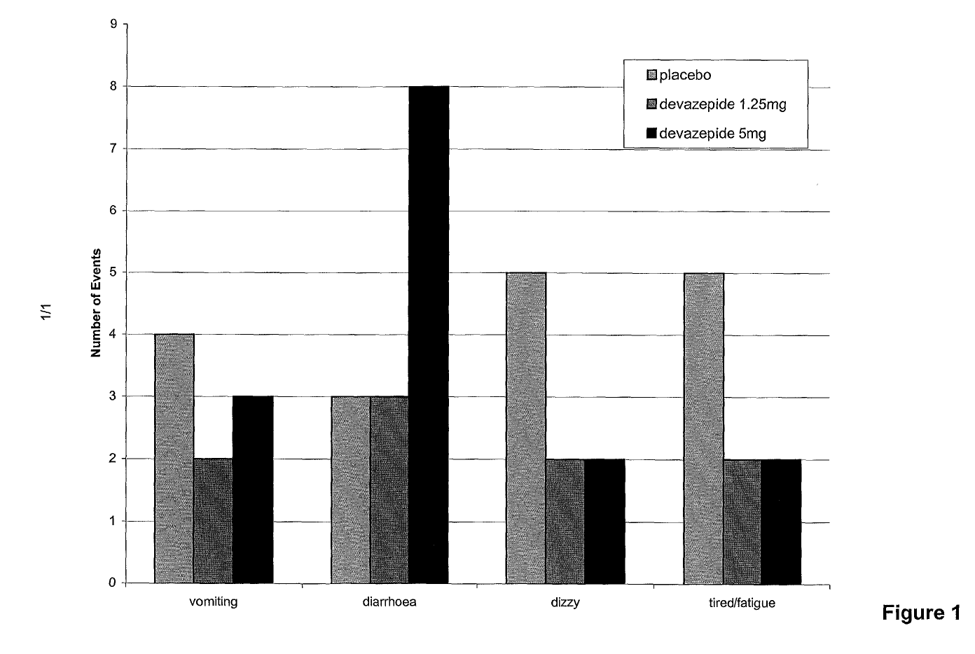

### METHOD OF TREATMENT

This invention relates to a novel method of treatment and to a novel use of a medicament.

International Patent Application No. WO 99/18967 describes pharmaceutical compositions for treating chronic and neuropathic pain which comprises an analgesic amount of an opioid and an opioid potentiating amount of a CCK antagonist. WO '967 describes the use of both CCK-A antagonists and CCK-B antagonists, although it is described that, generally, CCK-B antagonists are preferred. Moreover, page 2, lines 6 to 8 of WO '967 describes that CCK-A antagonists may be suitable, but only at relatively higher dosages.

One specific CCK-A antagonist which is mentioned in WO 99/18967 is devazepide, which is 3s-(-)1, 3-dihydro-3-(2-indolecarbonylamino)-1-methyl-5-phenyl-2H-1,4-benzodiazepin-2-one.

International Patent Application No. WO 99/18967 describes a pharmaceutical formulation comprising a CCK antagonist, such as devazepide (Devacade®), an opioid and a biphasic carrier, comprising a glyceride derivative organic phase. The CCK antagonist is intended to block the CCK receptors thereby reversing or preventing the development of opiate tolerance in patients and potentiating the analgesic effect of the opiate.

Administration of opioid analgesics is known to suffer from a number of disadvantages. In particular, opioids, such as morphine suffer from adverse reactions, including, inter alia, respiratory depression, circulatory depression, apnea, shock and cardiac arrest, being secondary to respiratory and/or circulatory depression. Other reactions include, constipation, nausea, vomiting, lightheadedness, dizziness, sedation, dysphoria, euphoria, cough suppression and sweating. Some of these effects seem to be more prominent in ambulatory patients and in those not experiencing severe pain. Some adverse reactions in ambulatory patients may be alleviated if the patient is in a supine position.

Less frequently observed reactions include the following:

body as a whole:—edema, antidiuretic effect, chills, muscle tremor, muscle rigidity;

cardiovascular:—flushing of the face, tachycardia, bradycardia, palpitation, faintness, syncope, hypotension, hypertension;

gastrointestinal:—dry mouth, biliary tract spasm, laryngospasm, anorexia, cramps, taste alterations;

genitourinary:—urine retention or hesitance, reduced libido and/or potency;

nervous system:—weakness, headache, agitation, tremor, uncoordinated muscle movements, seizure, paresthesia, alternations of mood (nervousness, apprehension, floating feelings), dreams, transient hallucination and disorientation, visual disturbances, insomnia, increased intracranial pressure;

skin:—pruritus, urticaria and other skin rashes; and

special senses:—blurred vision, nystagmus, diplopia, miosis, vertigo.

We have now surprisingly found that the administration of devazepide in combination with an opioid analgesic not only potentiates the analgesic effect of the opiate, but it advantageously mitigates the undesirable side effects of the opioid analgesic.

Thus, according to the invention we provide a method of treatment of a patient undergoing opioid analgesic therapy which comprises minimising or mitigating the side effects of the opioid by the administration of a therapeutically effective amount of devazepide.

According to a further feature of the invention we provide a method of treatment of a patient requiring analgesia which comprises the administration of a therapeutically effective amount of an opioid analgesic whilst minimising the side effects of the opioid by the separate, simultaneous or sequential administration of a therapeutically effective amount of devazepide.

In the method of the invention a variety of opioids may be used. Thus, the opioid may be selected from those which are effective analgesics and particularly those which need to be administered at relatively high or increasing doses. Examples include morphine, or a salt thereof such as the sulphate, chloride or hydrochloride, or the other 1,4-hydroxymorphinan opioid analgesics such as naloxone, meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these. Especially preferred analgesics which may be mentioned are hydromorphone, oxycodone, morphine, e.g. morphine sulphate and fentanyl. In a preferred embodiment of the invention the analgesic is morphine or morphine sulphate.

In the method of the invention the devazepide and/or the opioid may be administered using any methods conventionally known per se. Thus, such methods would include, but shall not be limited to, administration intravenously, orally, intrathecally, intranasally, intrarectally, intramuscularly/subcutaneously, by inhalation and by transdermal patch. Preferably, the opioid and/or devazepide are administered intravenously or orally. Oral administration is especially preferred. Preferentially, when the opioid and the devazepide will be administered using the same mode of administration. Thus, for example, when the opioid is administered intravenously then the devazepide will be administered intravenously also. Similarly, when the opioid is administered orally then the devazepide will be administered orally also. However, it is within the scope of the invention for either the opioid to be administered orally and the devazepide to be administered intravenously or vice versa.

In the method of the invention a variety of side effects may be inhibited, mitigated or minimised. Side effects of, e.g. morphine administration which may especially be mentioned include, but shall not be limited to, constipation, dizziness, tiredness/fatigue and vomiting.

Thus, in the method of the invention the daily dosage of devazepide may vary depending upon, inter alia, the weight of the patient, the method of administration, etc. Thus the daily dosage of devazepide may be up to 0.7 mg/kg/day. In patients that are suffering serious disorders, such as cancer patients, the weight of the patient may be very low and therefore the dosage of devazepide consequentially may be low. Thus, preferably, the daily dosage of devazepide may be from 25 \grave{1} g/kg/day to 0.7 mg/kg/day, more preferably from 50 \grave{1} g/kg/day to 0.5 mg/kg/day. For oral administration the daily dosage of devazepide may be from preferably 0.07 mg/kg/day to 0.29 mg/kg/day. For intravenous administration the dosage of devazepide is preferably 50 \grave{1} g/kg/day to 0.5 mg/kg/day.

In the method of the invention the dosage of the opioid analgesic administered may vary depending upon, inter alia, the nature of the opioid analgesic, the weight of the patient, the method of administration, etc. Thus, for example, the dosage of, e.g. an opioid, such as morphine, may be from 5 to 2000mg daily A particular dosage which may be mentioned is from 10 to 240mg daily. A daily dosage of morphine may be from 5 to 100mg or occasionally up to 500mg.

According to a further aspect of the invention we provide the use of devazepide in the manufacture of a pharmaceutical composition as hereinbefore described. In particular, we provide the use of devazepide in the manufacture of a medicament which inhibits or mitigates the undesirable side effects of administration of a therapeutically effective amount of an opioid analgesic.

We especially provide the use of devazepide in the manufacture of a medicament for use in the methods as hereinbefore described.

According to a further aspect of the invention we provide the use of devazepide in the manufacture of a medicament which inhibits or mitigates the undesirable side effects of administration of a therapeutically effective amount of an opioid analgesic.

The devazepide used in the method and/or the use of the invention is the S enantiomer. Preferentially, the S enantiomer wherein the level of R enantiomer, which may be present as an impurity, is not greater than 1.5% w/w.

The invention will now be illustrated by way of example only.

### EXAMPLE 1

### Clinical Crossover Study

A multi-centre, double blind placebo controlled crossover study was designed to investigate devazepide as adjunctive therapy to strong opioids in patients with moderate or severe neuropathic pain.

1.1 Rationale for Study Design

The study was conducted to observe the effects of two different doses for devazepide (1.25 mg b.d. and 5 mg b.d.) in patients when given as an adjunct to strong opioids. Previous studies have shown devazepide is well tolerated at levels up to 5 mg bd. This study was designed to further investigate the analgesic effect and the safety and toxicity of devazepide compared to placebo when administered twice daily for two weeks.

1.2 Pre-treatment

Patients completed a pre-treatment period of at least two weeks duration. During pre-treatment the patients continued to take stable, regular doses of strong opioids and completed an assessment of their pain in the evening prior to going to bed, using a pain questionnaire. Any adverse events, changes in concomitant medication and breakthrough analgesic used were recorded.

1.3 Treatment

Following the two-week pre-treatment period, each patient received blinded treatments of devazepide 1.25 mg, devazepide 5 mg and placebo twice daily for two weeks with washout periods between each treatment, according to the following schedule.

TABLE I
Treatment Schedule

| Study | | | | | | |
|---|---|---|---|---|---|---|
| period | Pre-treatment | Treatment 1 | Washout | Treatment 2 | Washout | Treatment 3 |
| Number | 2 weeks | 2 weeks | 4-21 days | 2 weeks | 4-21 days | 2 weeks |
| of weeks | | | | | | |
| or days | | | | | | |

On each day of treatment, assessment of pain, general activity and sleep interference were made by the patients and recorded on diary cards. At the end of each treatment period, the patient rated overall pain relief using a descriptive scale, and the opinion of both the patient and investigator of that treatment was recorded. All adverse events and concomitant medications were recorded throughout the study. Blood samples were collected at screening and at the end of each treatment for safety monitoring.

1.4 Results

The results are shown in Table I and Figure I.

TABLE I

| Complaint | placebo | devazepide 1.25 mg | devazepide 5 mg |
|---|---|---|---|
| diarrhoea | 3 | 3 | 8 |
| dizziness | 5 | 2 | 2 |
| tired/fatigue | 5 | 2 | 2 |
| vomiting | 4 | 2 | 3 |

1. A method of treatment of a patient undergoing opioid analgesic therapy which comprises minimising or mitigating the side effects of the opioid by the administration of a therapeutically effective amount of devazepide.

2. A method of treatment of a patient requiring analgesia which comprises the administration of a therapeutically effective amount of an opioid analgesic whilst minimising the side effects of the opioid by the separate, simultaneous or sequential administration of a therapeutically effective amount of devazepide.

3. A method according to claim 1 characterised in that the opioid is selected from the group morphine, or a salt thereof such as the sulphate, chloride or hydrochloride, or the other 1,4-hydroxymorphinan opioid analgesics such as naloxone, meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these.

4. A method according to claim 2 characterised in that the opioid is selected from the group morphine, or a salt thereof such as the sulphate, chloride or hydrochloride, or the other 1,4-hydroxymorphinan opioid analgesics such as naloxone, meperidine, butorphanol or pentazocine, or morphine-6-glucuronide, codeine, dihydrocodeine, diamorphine, dextropropoxyphene, pethidine, fentanyl, alfentanil, alphaprodine, buprenorphine, dextromoramide, diphenoxylate, dipipanone, heroin (diacetylmorphine), hydrocodone (dihydrocodeinone), hydromorphone (dihydromorphinone), levorphanol, meptazinol, methadone, metopon (methyldihydromorphinone), nalbuphine, oxycodone (dihydrohydroxycodeinone), oxymorphone (dihydrohydroxymorphinone), phenadoxone, phenazocine, remifentanil, tramadol, or a salt of any of these

5. A method according to claim 3 characterised in that the opioid is selected from the group hydromorphone, oxycodone, morphine and fentanyl.

6. A method according to claim 4 characterised in that the opioid is selected from the group hydromorphone, oxycodone, morphine and fentanyl.

7. A method according to claim 5 characterised in that the opioid is selected from the group morphine and morphine sulphate.

8. A method according to claim 6 characterised in that the opioid is selected from the group morphine and morphine sulphate.

9. A method according to claim 1 characterised in that the method of delivery of the devazepide and/or the opioid is selected from the group, administration intravenously, orally, intrathecally, intranasally, intrarectally, intramuscularly/subcutaneously, by inhalation and by transdermal patch.

10. A method according to claim 2 characterised in that the method of delivery of the devazepide and/or the opioid is selected from the group, administration intravenously, orally, intrathecally, intranasally, intrarectally, intramuscularly/subcutaneously, by inhalation and by transdermal patch.

11. A method according to claim 9 characterised in that the devazepide is administered intravenously or orally.

12. A method according to claim 10 characterised in that the devazepide is administered intravenously or orally.

13. A method according to claim 11 characterised in that the devazepide is administered orally.

14. A method according to claim 12 characterised in that the devazepide is administered orally.

15. A method according to claim 9 characterised in that the opioid is administered intravenously and the devazepide is administered intravenously.

16. A method according to claim 10 characterised in that the opioid is administered intravenously and the devazepide is administered intravenously.

17. A method according to claim 9 characterised in that the opioid is administered orally and the devazepide is administered orally.

18. A method according to claim 10 characterised in that the opioid is administered orally and the devazepide is administered orally.

19. A method according to claim 9 characterised in that the opioid is administered by intravenous administration or oral administration.

20. A method according to claim 10 characterised in that the opioid is administered by intravenous administration or oral administration.

21. A method according to claim 1 characterised in that the daily dosage of devazepide is up to 0.7 mg/kg/day.

22. A method according to claim 2 characterised in that the daily dosage of devazepide is up to 0.7 mg/kg/day.

23. A method according to claims 21 characterised in that the daily dosage of devazepide is from 25 μg/kg/day to 0.7 mg/kg/day.

24. A method according to claims 22 characterised in that the daily dosage of devazepide is from 25 μg/kg/day to 0.7 mg/kg/day.

25. A method according to claim 23 characterised in that the daily dosage of devazepide is from 50 μg/kg/day to 0.5 mg/kg/day.

26. A method according to claim 24 characterised in that the daily dosage of devazepide is from 50 μg/kg/day to 0.5 mg/kg/day.

27. A method according to claim 25 characterised in that the devazepide is administered orally and the daily dosage of devazepide is from 0.07 mg/kg/day to 0.29 mg/kg/day.

28. A method according to claim 26 characterised in that the devazepide is administered orally and the daily dosage of devazepide is from 0.07 mg/kg/day to 0.29 mg/kg/day.

29. A method according to either of claims 25 characterised in that the devazepide is administered intravenously administration the dosage of devazepide is from 50 μg/kg/day to 0.5 mg/kg/day.

30. A method according to either of claims 26 characterised in that the devazepide is administered intravenously administration the dosage of devazepide is from 50 μg/kg/day to 0.5 mg/kg/day.

31. A method according to claim 1 characterised in that the daily dosage of the opioid is from 5 to 2000 mg daily.

32. A method according to claim 2 characterised in that the daily dosage of the opioid is from 5 to 2000 mg daily.

33. A method according to claim 31 characterised in that the daily dosage of the opioid is from 5 to 100 mg daily.

34. A method according to claim 32 characterised in that the daily dosage of the opioid is from 5 to 100 mg daily.

35. A method according to claim 1 characterised in that the side effect which is inhibited, mitigated or minimised is selected from the group, constipation, dizziness, tiredness/fatigue and vomiting.

36. A method according to claim 2 characterised in that the side effect which is inhibited, mitigated or minimised is selected from the group, constipation, dizziness, tiredness/fatigue and vomiting.

37. A method according to claim 1 characterised in that the devazepide used is the S enantiomer wherein the level of R enantiomer is not greater than 1.5% w/w.

38. A method according to claim 2 characterised in that the devazepide used is the S enantiomer wherein the level of R enantiomer is not greater than 1.5% w/w.

39. The use of devazepide in the manufacture of a medicament which inhibits or mitigates the undesirable side effects of administration of a therapeutically effective amount of an opioid analgesic.

40. The use according to claim 39 characterised in that the devazepide used is the S enantiomer wherein the level of R enantiomer is not greater than 1.5% w/w.

41. The use of devazepide in the manufacture of a medicament for use in the method of either of claim 1.

42. The use of devazepide in the manufacture of a medicament for use in the method of either of claim 2.
